(19) United States
(12) Patent Application Publication
(10) Pub. No.: US 2005/0043623 A1
(43) Pub. Date: Feb. 24, 2005

(54) Method and measuring device for examining a compressible tissue

(76) Inventors: Jukka Jurvelin, Kuopio (FI); Ilkka Kiviranta, Kuopio (FI); Juha Toyras, Kuopio (FI)

Correspondence Address: YOUNG & THOMPSON, 745 SOUTH 23RD STREET, 2ND FLOOR, ARLINGTON, VA, 22202, US

(21) Appl. No.: 10/491,345

(22) PCT Filed: Apr. 23, 2002
(86) PCT No.: PCT/FI02/00334

(30) Foreign Application Priority Data
Apr. 23, 2001 (FI) 20010832

(51) Int. Cl. A61B 8/00

(52) U.S. Cl. 600/449

(57) ABSTRACT

A measuring method and a measuring device for examining a compressible tissue, such as articular cartilage, said measuring device comprising an elongated rigid frame (1), a measuring arm (2) attached to the frame and preferably comprising a contact surface (3) to be placed against the tissue to be examined, and a measuring stud (4) and means (5) for processing signals obtained from the measuring stud. The measuring stud is an ultrasound probe for emitting ultrasound into the tissue to be examined and receiving ultrasound from the tissue to be examined.

The present invention relates to a method as defined in the preambles of claims 1 and 6 and to a measuring device as defined in the preamble of claim 8 for examining a compressible tissue, such as articular cartilage.

Articular cartilage consists of differentiated connective tissue that contains no blood vessels, lymphatic vessels or nerves. Articular cartilage is the stiffest kind of soft tissue in the human body, yet it is clearly softer than bone. The thickness of articular cartilage varies in different joints from a few micrometers to several millimeters. The thickness of articular cartilage may also be different in different parts of joint surfaces. Articular cartilage has two main functions—reducing the stress applied to the subchondral bone and reducing friction in the joint.

The properties of articular cartilage, including mechanical properties, undergo changes during articular cartilage diseases such as arthrosis, rheumatoid arthritis and chondromalacia. Softening of cartilage is often the first perceptible symptom of cartilage decay. On the other hand, e.g. after a corrective treatment of an articular cartilage injury by cellular transfer, the corrective tissue will gradually solidify, allowing measurement of mechanical properties of the tissue to be used for the estimation of the results of surgery.

The simplest method used for estimating the stiffness of articular cartilage is to feel the cartilage surface by pressing on it with a metallic instrument in connection with an operation. However, the results of such measurement are highly subjective and inconsistent.

Various measuring devices have been developed for use in connection with surgical operations. Prior-art technology in the field of the invention is primarily represented by patent FI 90616. A tissue stiffness measuring device disclosed in this specification comprises an elongated rigid frame comprising a contact surface which, in connection with a measurement, is pressed against the surface of the tissue being examined. The frame supports a measuring arm provided with a projecting measuring stud or an equivalent, relatively small pin. The measuring arm is also provided with a sensor for the measurement of the stress applied to it via the measuring stud from the tissue being measured.

When this measuring device is used, its contact surface is pressed against the tissue to be measured so that the measuring stud projecting outside the contact surface compresses the cartilage under it. Thus, the cartilage exerts a pressure proportional to its stiffness on the measuring stud and the measuring arm. This pressure produces measurable stresses in the measuring arm. Corresponding measurable stresses are produced in the frame when the contact surface is pressed against the cartilage to be measured.

This prior-art measuring device has proved to be a very workable and effective tool in the examination of compressible tissues, such as articular cartilage. The main problem with this prior-art device is only the scantness of information produced by it. The device was so designed that the effect of cartilage thickness could be minimized, because the device could not be used for the measurement of cartilage thickness. In investigations concerning cartilage tissue, there is clearly a need to obtain other information about the tissue in addition to its stiffness, which does not tell all about the tissue being examined. Thus, a need has arisen to develop a measuring device that can be used to accomplish, in addition to arthroscopic mechanical stiffness measurement of the tissue, a more detailed characterization of the tissue structure.

As for the features characteristic of the invention, reference is made to the claims.

In the method of the invention, when a stiff tissue is examined via arthroscopic indentation measurement, the compression of the tissue is not effected using a prior-art measuring stud but by means of an ultrasound probe, the thickness and compression of the tissue being measured by ultrasound while the compressive force is measured by a known technique using a strain gauge.

Thus, in the method of the invention, it is possible to determine the absolute dynamic or static Young's modulus and the modulus of shear. Young's modulus describes the elastic stiffness of articular cartilage under stress and is therefore a good indicator of mechanical functionability. Like Young's modulus, the modulus of shear is also an effective indicator of mechanical functionability.

In addition, the method can be used to determine the relaxation speed, i.e. reduction of dynamic stiffness of a tissue and its indentation stiffness dependent on duration of stress as well as other possible quantitative parameters descriptive of the dynamic behavior of articular cartilage. These parameters take into account the relaxation behavior typical of articular cartilage and they are therefore indicators of the mechanical properties of cartilage under long-time stress. They have also been found to be predictive of changes in cartilage composition, especially in proteoglycane content, which is an important biochemical component of cartilage.

In another method according to the invention, to determine structural properties of the tissue, an ultrasound probe is held at a distance from the tissue surface, preferably inside the frame or arm of the measuring device so that there remains between the ultrasound probe and the tissue surface some physiologic salt solution, with which the joint to be examined has been filled in connection with arthroscopy. In this way, using an ultrasound probe, pulse-echo measurements can be made on the tissue surface, interior portions of the tissue and/or on the bone under the tissue.

During measurement, the ultrasound probe can be kept stationary, producing result data about a given point of the tissue. However, the measurements are performed by moving the ultrasound probe, i.e. by scanning a certain tissue surface with ultrasound.

By examining e.g. articular cartilage in this manner, it is possible to determine the thickness of the cartilage layer, the ability of the cartilage surface, internal structures and subchondral bone to reflect or scatter the sound as well as the attenuation of ultrasound as a function of frequency.

Of the above-mentioned acoustic parameters:
Cartilage layer thickness is understandably an important parameter in an estimation of the condition of cartilage.
The ability of cartilage surface and internal structures of cartilage to reflect/scatter ultrasound are a sensitive indication of cartilage quality and especially of the condition of collagenous fibers on cartilage surface.
There is no wide experience about the application of frequency-dependent attenuation to the estimation of cartilage condition, but this parameter (BUA) has proved to be useful in the estimation of bone quality. It may be useful in the estimation of cartilage quality as well.
Back-scattering parameters have been successfully applied in the estimation of both cartilage and bone quality.
The ability of subchondral bone to reflect/scatter ultrasound is a sensitive indication of bone quality.

The device of the invention for the measurement of compressible tissue, such as articular cartilage, comprises an elongated rigid frame and a measuring arm supported by the frame. Th arm can additionally be provided with a contact surface to be placed against the tissue to be examined. The device further comprises a measuring stud and means for processing signals obtained from the measuring stud. According to the invention, the measuring stud is an ultrasound probe for emitting ultrasound into the tissue to be examined and for receiving ultrasound from the tissue to be examined.

In an embodiment of the invention, the measuring stud is an element projecting from the measuring arm. The measuring arm preferably comprises a sensor for measuring the force applied to the measuring arm from the tissue to be examined when the measuring stud is being pressed against the tissue to be examined. Thus, the force applied to press the probe against the tissue is measured by means of the sensors provided in the measuring device while the probe is simultaneously emitting an ultrasound signal and gathering information about the tissue via the signal.

In an embodiment of the invention, the measuring device comprises shifting means for shifting the position of the ultrasound probe between a projecting outer position against the surface of the tissue to be examined and an inner position at a distance from the surface of the tissue.

The shifting means for shifting the position of the ultrasound probe between an outer position and an inner position preferably comprise elements by means of which the ultrasound probe can be moved relative to the frame of the measuring device e.g. along suitable guide bars. However, another possibility is that the ultrasound probe is fixedly mounted and a movable contact surface or equivalent is provided near the probe, e.g. around it. By moving the contact surface, the projection of the probe can be changed and it can also be brought to a position completely inside the outermost surface defined by the contact surface.

In an embodiment of the invention, the measuring device comprises scanning means for moving the ultrasound probe inside the measuring arm and surveying a given area of the tissue by ultrasound emitted by the ultrasound probe. The scanning motion is performed at a known distance so that the ultrasound echo obtained can be correctly interpreted and that the results are comparable. This known distance may be a constant distance or it may be varied provided that it is known exactly as a function of time or position.

The shifting means and the scanning means are preferably formed by the same structure, in other words, the means used to retract the ultrasound probe to a position inside the measuring device also comprises a function that makes it possible to use it in this inner position to move the probe to perform a desired scanning movement. By passing the probe over the tissue, an ultrasound image of the tissue covered by the scan can be produced. The scanning movement itself may consist of a linear, rotating, curved or similar motion, mainly depending on the dimensions and geometry of the internal frame part and the probe. Another possibility is that the scanning movement is identical with the movement of retraction of the ultrasound probe into the measuring device or its movement out of the measuring device.

In an embodiment of the invention, the ultrasound probe used may consist of an ultrasound crystal matrix, i.e. a set of ultrasonic probes that together form an area covering all or part of the tissue to be examined. Thus, instead of a scan, the probe matrix can provide information about a desired area of the tissue.

The method and measuring device of the invention can be successfully used for quantitative determination of mechanical and structural properties of articular cartilage in connection with arthroscopy. Extending acoustic measurements even to the bone under the cartilage allows quantitative determination of the properties of the subchondral bone. It is also very important to know the properties of the subchondral bone because changes in said properties have been found to bear a relation to degeneration of cartilage.

The method and measuring device of the invention are particularly advantageous in measurements of the echo reflected from a cartilage-bone interface, which provides cartilage thickness and compression information as well as, on the basis of force measurements obtained, material parameters descriptive of the tissue.

After the measurements according to the invention, an actual analysis and diagnosis can be made by combining all measured parameters and the information obtained from them. A diagnosis is preferably made by collecting extensive reference material about all corresponding measurements to be performed and entering the new measured data into such a file containing reference material, whereupon it is possible to obtain an estimate of the condition of the joint on the basis of the previously collected reference material.

The method and measuring device of the invention have significant advantages as compared with prior art. The invention makes it possible to achieve measurement of actual material properties of cartilage, measurement of structural and compositional properties of cartilage and subchondral bone and measurement of cartilage thickness. Moreover, variations in cartilage thickness have no effect on the measurement results as in prior-art methods.

In the following, the invention will be described in detail with reference to the attached drawings, wherein

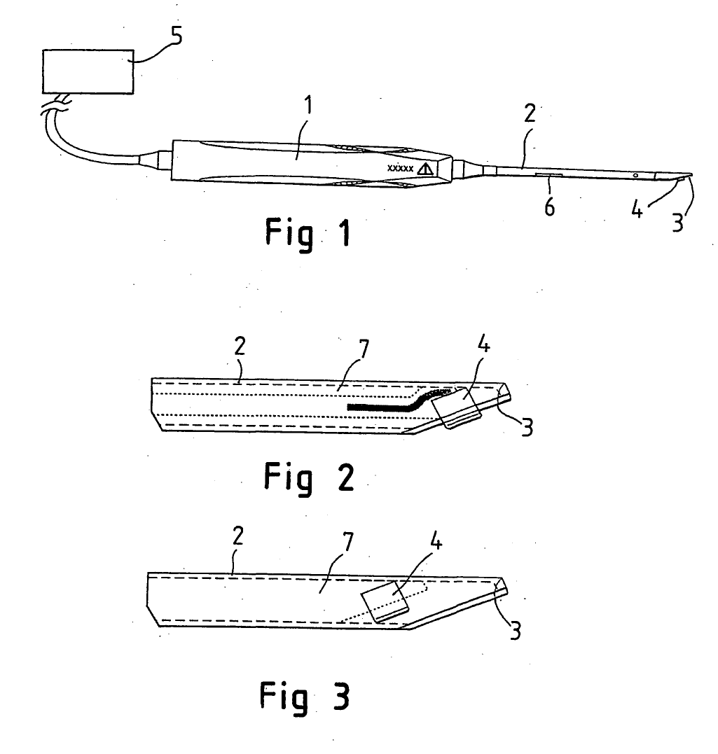

FIG. 1 presents a general view of a measuring device according to the invention,

FIG. 2 presents a magnified sectioned view of the tip of the measuring device in FIG. 1 in a first measuring position,

FIG. 3 presents a magnified sectioned view of the tip of the measuring device in FIG. 1 in a second measuring position,

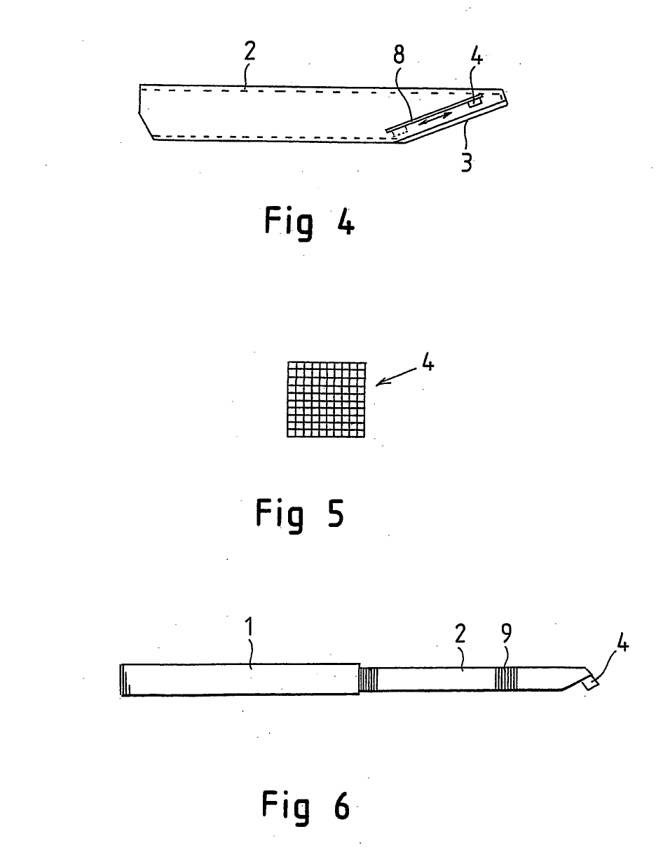

FIG. 4 presents a sectioned view of a second embodiment of the structure of the tip of the measuring device,

FIG. 5 illustrates an ultrasound crystal structure used in the invention, and

FIG. 6 presents a general view of a second measuring device according to the invention.

The measuring device presented in FIG. 1 comprises an elongated, rigid frame 1, i.e. a handle, and a straight and rigid measuring arm 2 attached to the frame. The beveled outer end of the measuring arm comprises a contact surface 3 to be placed against the tissue to be examined, with a measuring stud 4 protruding from this surface. In addition, the measuring arm is provided with at least one sensor, preferably a strain gauge, for measuring the force applied via the measuring stud from the tissue being examined to the measuring arm. The measuring device is connected to suitable means 5 for processing the signals detected by the measuring arm.

The measuring stud 4 is an ultrasound probe and it is presented in greater detail in FIGS. 2 and 3. In the first measuring position shown in FIG. 2, the ultrasound probe 4 is in a projecting outer position. Thus, the ultrasound probe can be used to measure mechanical properties of a tissue, such as cartilage, by pressing it against the tissue. During the measurement, cartilage thickness and compression are measured by ultrasound and the force applied to produce the compression is measured by one or more strain gauges 6.

In the second measuring position shown in FIG. 3, the ultrasound probe 4 has been retracted by means of suitable shifting means 7 into an inner position, where it is inside the measuring arm at a distance from the oblique contact surface 3 of the measuring arm. In this embodiment, ‘shifting means’ refers to an interior part of the measuring arm 2, the ultrasound probe 4 being attached to said interior part so that it can be moved inside the measuring arm in its longitudinal direction. When the oblique contact surface 3 is pressed against the tissue to be examined, there remains between the tissue surface and the probe 4 a gap of constant size filled with physiologic salt solution. After this, it is possible to perform several pulse-echo measurements on the joint surface and obtain information as described above about the cartilage, the cartilage-bone interface and the internal parts of the bone.

The ultrasound probe 4 is preferably so mounted and arranged inside the measuring arm 2 that it can be moved during the measurement at a known distance relative to the surface of the tissue to be measured. Thus, by passing the probe across a given area of the tissue, the tissue area concerned or the area under the tissue can be accurately imaged, i.e. an ultrasound image of this area can be produced. By maintaining a constant distance of the probe during all scanning movements over different surface areas of the same tissue, measurement results that are comparable and readily interpretable are obtained. On the other hand, it will suffice to keep the probe at a known, constant or knowably varying distance, because in this case the results can be rendered comparable by computational means.

If the ultrasound probe is not moved but only held in a projecting position as in FIG. 1 or 2, point-specific information about the tissue can be obtained by pressing the probe 4 into the tissue. Thus, a device according to the invention may be an embodiment as illustrated in FIGS. 1 and 2, wherein the ultrasound probe 4 is fixedly mounted and continuously protruding out of the oblique contact surface 3. In this case, the tissue to be examined will return a point-specific acoustic signal that provides significant information especially about the bone and the bone interface in the form of possible fading or enhancement of reflection or scattering.

In the embodiment in FIG. 4, the ultrasound probe 4 is attached to scanning means 8, i.e. to a guide bar along which the probe can be moved back and forth between its extreme positions shown in the figure. The guide bar 8 extends at a constant distance from the oblique contact surface 3 at the end of the measuring arm 2. Thus, when a measurement is being performed and the contact surface 3 is pressed against the tissue to be examined, the ultrasound probe 4 moves at a constant distance from the tissue. The motion of the probe along the guide bar can be implemented e.g. by mechanical, electric, pneumatic or hydraulic means.

In the embodiment presented in FIG. 5, the ultrasound probe 4 is an ultrasound crystal matrix consisting of a plurality of separate ultrasound crystals. The matrix covers the tissue area to be examined at a time and the matrix is not moved during the examination. The measurement is performed one matrix element or crystal at a time so that measurements by different crystals do not interfere with each other. Thus, point-specific measurements uniformly covering the entire matrix area of the tissue to be examined are obtained.

In the embodiment presented in FIG. 6, the measuring device is a pen-like structure in which a rigid frame 1 and a thinner measuring arm 2 extending from it are connected together. The end of the measuring arm is provided with an ultrasound probe 4 mounted in an oblique position relative to the longitudinal direction of the arm. There is no contact surface at the end of the measuring arm, but the device is used by only pressing the probe against the tissue to be examined. Placed in the measuring arm relatively close to the probe 4 are strain gauges or equivalent sensors 9 for measuring the force applied to press the ultrasound probe against the tissue. Thus, the device produces point-like measurement results for the tissue to be examined.

Thus, when the device of the invention is used to examine tissue such as cartilage and subchondral bone, both mechanical, structural and compositional properties of the tissue can be determined by the same device in connection with arthroscopy.

1. Method for examining a compressible tissue, such as articular cartilage, via arthroscopic indentation measurement, characterized in that the method comprises pressing the tissue to be examined with an ultrasound probe, measuring the thickness and compression of the tissue by means of ultrasound emitted by the probe, and measuring the compressive force applied.

2. Method according to claims 1, characterized in that the compressive force applied is measured by means of a strain gauge.

3. Method according to claim 1, characterized in that stiffness moduli, such as Young's modulus and shear modulus, are determined for the tissue by a fast measurement.

4. Method according to claim 1, characterized in that the reduction of dynamic stiffness, i.e. relaxation speed and indentation stiffness are determined via a long-time measurement keeping either the compressive force or the compression constant.

5. Method according to claim 4, characterized in that the indentation stiffness is measured after the lapse of a certain length of time or in a state of balance.

6. Method for examining a compressible tissue, such as articular cartilage, via arthroscopic indentation measurement, characterized in that an ultrasound probe is held at a distance from the tissue surface to be examined so that some physiologic salt solution remains between the ultrasound probe and the tissue, whereupon pulse-echo measurements on the tissue surface, interior portions of the tissue and/or the bone under it are performed.

7. Method according to claim 6, characterized in that, using ultrasound emitted by the ultrasound probe, a scan across the tissue surface to be examined is made by moving the ultrasound probe.

8. Measuring device for examining a compressible tissue, such as articular cartilage, said device comprising an elongated rigid frame (1), a measuring arm (2) attached to the frame and a measuring stud (4) and means (5) for processing signals obtained from the measuring stud, characterized in that the measuring stud (4) is an ultrasound probe for emitting ultrasound into the tissue to be examined and receiving ultrasound from the tissue to be examined.

9. Measuring device according to claim 8, characterized in that the measuring device comprises a contact surface (3) that can be placed against the tissue to be examined.

10. Measuring device according to claim 8, characterized in that the ultrasound probe (4) is an element projecting from the contact surface and the measuring arm comprises a sensor (6) for measuring the force applied to the measuring arm via the ultrasound probe from the tissue to be examined.

11. Measuring device according to claim 10, characterized in that the measuring device comprises shifting means (7) for shifting the position of the ultrasound probe between a projecting outer position against the surface of the tissue to be examined and an inner position at a distance from the surface of the tissue.

12. Measuring device according to claim 8, characterized in that the ultrasound probe (4) is placed inside the measuring arm and attached to scanning means (8) for moving the ultrasound probe at a known distance from the tissue to be examined and surveying a given area of the tissue by ultrasound emitted by the ultrasound probe.

13. Measuring device according to claim 12, characterized in that the known distance is constant.

14. Measuring device according to claim 12, characterized in that the known distance is variable.

15. Measuring device according to claim 11, characterized in that the shifting means are so implemented that they also function as scanning means for moving the ultrasound probe.

16. Measuring device according to claim 8, characterized in that the ultrasound probe consists of an ultrasound crystal matrix that substantially covers the tissue area to be examined.
